(19) United States
(12) Patent Application Publication
(10) Pub. No.: US 2008/0306362 A1
(43) Pub. Date: Dec. 11, 2008

(54) DEVICE AND SYSTEM FOR MONITORING CONTENTS OF PERSPIRATION

(76) Inventor: Owen Davis, New York, NY (US)

Correspondence Address: Seth H. Ostrow, Esq.;DREIER LLP, 12th Floor, 499 Park Avenue, New York, NY, 10022, US

(21) Appl. No.: 11/758,370

(22) Filed: Jun. 5, 2007

(51) Int. Cl. A61B 5/00

(52) U.S. Cl. 600/307; 702/19

(57) ABSTRACT

Described are devices and systems for monitoring a content of perspiration. The device comprises a memory storing an index of values corresponding to amounts of a content in perspiration, a detection arrangement detecting the content in the perspiration, a processing unit determining an amount of the content in the perspiration, and a feedback arrangement providing feedback data indicative of a value corresponding to the determined amount.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### BACKGROUND OF THE INVENTION

While physicians can detect symptoms of medical conditions through non-invasive means (e.g., patient questioning, palpation, superficial examination), it is often likely that the patient will have to undergo an invasive procedure to further analyze or confirm a physician's initial diagnosis. The invasive procedure may be a simple, out-patient procedure such as giving blood or may require a hospital stay, requiring recovery time and post-procedure monitoring if the procedure is more complicated (e.g,. a biopsy). Unless the physician detects the symptoms during a regularly-scheduled medical exam, the patient may not visit the physician until the symptoms have manifested themselves, which can effect a seriousness of the condition and/or an aggressiveness of a treatment protocol.

For example, dehydration is a medical condition which can generally be rectified by ingesting or infusing fluids and/or stopping fluid loss. However, these remedies are typically administered after the dehydration has manifested itself as one or more symptoms, e.g., headaches, sporadic vision problems, decreased blood pressure (hypotension), dizziness or fainting when standing up, etc. In extreme cases, dehydration can lead to delirium, unconsciousness and, potentially, death.

Individuals can avoid dehydration by drinking an amount of water commensurate with their body types, medical conditions and/or physical activities. For example, it is suggested that athletes drink water before, during and after practice and contests, because perspiration accelerates a normal water loss rate of the body. However, the only feedback an individual typically receives about his hydration is one or more of the symptoms of dehydration. Thus, the individual is limited to being reactive to his hydration level.

### SUMMARY OF THE INVENTION

The present invention generally relates to devices and systems for monitoring contents of perspiration. An exemplary embodiment of a device comprises a memory storing an index of values corresponding to amounts of a content in perspiration, a detection arrangement detecting the content in the perspiration, a processing unit determining an amount of the content in the perspiration, and a feedback arrangement providing feedback data indicative of a value corresponding to the determined amount. The device may further comprise a sweating agent inducing the perspiration when applied to skin. The sweating agent may be pilocarpine. The device may further comprise a housing having first and second faces, the sweating agent being disposed on the first face. The second face may include an engagement arrangement for removably engaging at least one of the memory, the processing unit and the feedback arrangement. The content may be a salt, an alcohol or a mineral.

The device may further comprise an adhesive for coupling to skin. The detection arrangement may comprise electrical contacts delivering a current to the perspiration. The processing unit may compute a conductivity of the perspiration as a function of the current. The values in the index may correspond to conductivities and hydration levels, and the feedback data may indicate the hydration level.

Another exemplary embodiment of the device may include a sensor as the detection arrangement measuring the amount of the content in the perspiration. The sensor may transmit a signal to the processing unit indicative of the amount. The device may further include a power source such as, for example, an alkaline battery, a rechargeable battery and a solar cell.

The feedback arrangement may include at least one of a liquid crystal display, a light-emitting diode, an audible alarm, a mechanical alarm and a wireless communication arrangement. The feedback data of the wireless communication arrangement may be included in a wireless signal transmitted thereby.

In one exemplary embodiment, a predetermined value from the index of values is a threshold value. The feedback arrangement may provide further feedback data indicating that the value is one of greater and less than the threshold value.

An exemplary embodiment of a system according to the invention may include a computing device and a wearable measuring device measuring an amount of a content of perspiration and transmitting feedback data indicative of the amount to the computing device. The wearable measuring device may include a memory storing an index of values corresponding to amounts of a content in perspiration, a detection arrangement detecting the content in the perspiration and a processing unit determining an amount of the content in the perspiration and generating the feedback data as a function of the amount. The computing device may be a server, a mobile phone, a PDA, a portable media player, a portable gaming device, a handheld computer, etc. The content may be a salt, an alcohol, a mineral or any other content of perspiration.

The system may further comprise a database storing the feedback data. The system may further comprise a servo system, wherein one of the computing device and the wearable measuring device transmit the feedback data to the servo system and the servo system executes a predetermined operation upon receipt of the feedback data. The servo system may be, for example, an intravenous fluid delivery system or a vehicle ignition system.

The wearable measuring device may include a unique identifier and transmit the unique identifier to the computing device. The computing device may associate the feedback data with the unique identifier.

In another exemplary embodiment, the device may include a memory storing a first value corresponding to a conductivity of perspiration, a power source supplying a current to the perspiration, a processing unit generating a second value as a function of the current, the processing unit comparing the first and second values to determine a hydration level, and a feedback arrangement providing feedback indicative of the hydration level. The feedback may be a visual indicator and an audible indicator. The device may further include a housing encasing the memory, the power source, the processing unit and the feedback arrangement, and a sweating agent disposed on the housing to induce the perspiration.

In a further exemplary embodiment, the device may include a memory storing a first value corresponding to a predetermined amount of a content of perspiration, a sensor measuring an amount of the content of the perspiration, a processing unit generating a second value as a function of the measured amount, the processing unit comparing the first and second values to determine a hydration level, and a feedback arrangement providing feedback indicative of the hydration level. The feedback may be a visual indicator and an audible indicator. The device may further include a housing encasing the memory, the power source, the processing unit and the feedback arrangement, and a sweating agent disposed on the housing to induce the perspiration.

### BRIEF DESCRIPTION OF THE DRAWINGS

The invention is illustrated in the figures of the accompanying drawings which are meant to be exemplary and not limiting, in which like references are intended to refer to like or corresponding parts, and in which:

FIG. 1 shows an exemplary embodiment of a device for monitoring hydration according to the present invention;

FIG. 2 shows an exemplary embodiment of an internal surface of a device for monitoring hydration according to the present invention; and

FIG. 3 shows an exemplary embodiment of a system for monitoring hydration according to the present invention.

### DETAILED DESCRIPTION OF THE PREFERRED EMBODIMENTS

In the following description of the preferred embodiments, reference is made to the accompanying drawings that form a part hereof, and in which is shown by way of illustration exemplary embodiments in which the invention may be practiced. It is to be understood that other embodiments may be utilized and structural changes may be made without departing from the scope of the present invention.

FIG. 1 shows an exemplary embodiment of a device 100 for analyzing a component of perspiration to, for example, monitor a hydration level, an alcohol level, a mineral level, a salt level, etc. of a user. In the exemplary embodiment, the device 100 may be formed as a patch which can be applied to a user's skin. The patch may be utilized as a stand-alone device, incorporated into a wearable article (e.g., a wristband, a watch, etc.) or utilized in combination with the wearable article or handheld device, as described below. Generally, the device 100 may be any shape and/or size and be manufactured from any one or more of various materials (e.g., fabrics, polymers, rubbers, hydrogels, plastics, ceramics, etc.). Preferably, the device 100 is lightweight and maintains a low profile such that the user does not bear a burden while it is being worn. Additionally, the device 100 may be utilized as a single-use item (e.g., disposable after being worn a single time) or as a reusable item which can be cleaned and/or sterilized between uses.

In the exemplary embodiment shown in FIG. 1, the device 100 includes a housing 110 which may be coupled to the individual's skin via, for example, an adhesive layer 115 formed on a selected portion of an internal face of the housing 110. As such, the device 100 may be adhered to a backing and separated therefrom when applied to the user's skin. In this embodiment, the device 100 may be sized and shaped to be coupled to any part of the user's body. For example, as noted above, the device 100 may be formed as a patch which can attach to the user's arm or legs. Alternatively, the device 100 can be formed as a tip (or another predefined shape) which couples to the user's fingertips. That is, as understood by those of skill in the art, the finger tips are one of the most porous areas on the body, and thus, may be a good place to harvest perspiration. In other exemplary embodiments, the housing 110 may be formed from tape (e.g., double-sided), a strap or a pocket.

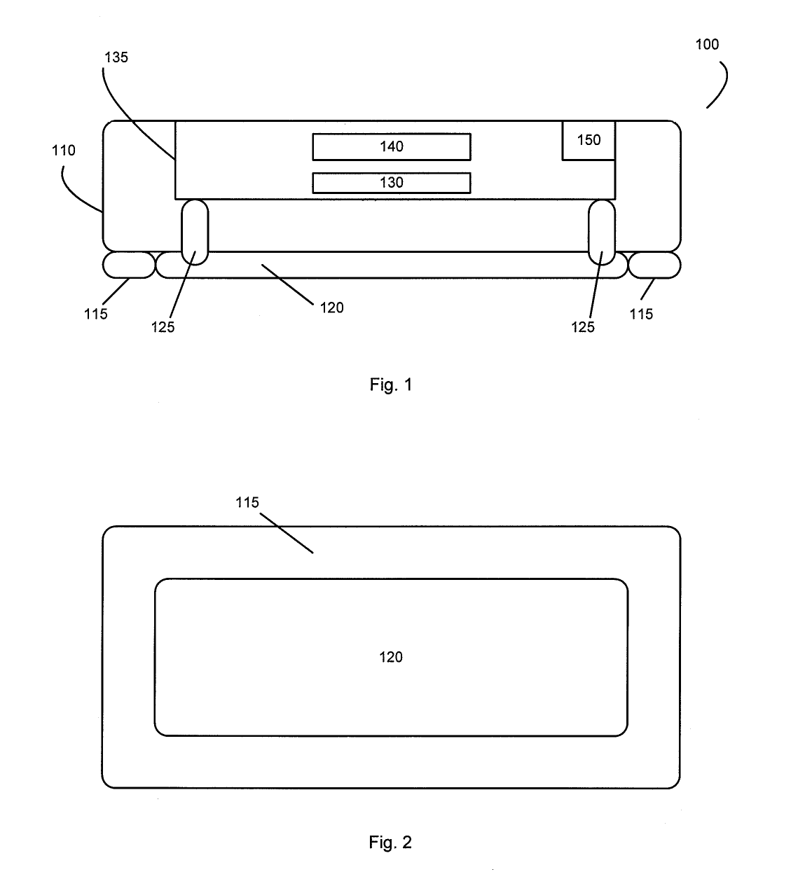

The device 100 may further include a sweating agent 120 which is disposed on the internal face of the housing 110. FIG. 2 shows an exemplary embodiment of the internal face of the device 100. Although, the terms “internal” and “external” may be used herein to describe locations/positions of relative elements of the device 100, those of skill in the art will understand that these terms do not indicate absolute position. For example, when the device 100 is applied to the user, the internal face of the housing 110 may be that which faces the user's skin with an external face facing away therefrom.

As shown in FIG. 2, the adhesive layer 115 may be formed as a perimeter around the sweating agent 120, allowing the sweating agent 120 to contact the skin directly. Alternatively, the adhesive layer 115 may be painted over a portion of the internal face of the device 100, allowing re-application as the device 100 is reused. In this exemplary embodiment, the adhesive selected for the adhesive layer 115 is preferably water permeable, allowing sweat to soak therethrough into the sweating agent 120, as described further below.

When the device 100 is applied to the user's skin, the sweating agent 120 may induce perspiration. For example, in a clinical setting, the user may be a patient in a hospital bed. Although not engaging in activity which would result in perspiration, a local area of the user's skin covered by the device 100 may produce sweat due to the sweating agent 120. The sweating agent 120 may be manufactured from, for example, pilocarpine or any other material which induces perspiration and may also possess an absorptive ability to retain at least some of the sweat that is excreted. In another exemplary embodiment, a reservoir may be formed between the housing 110 and the sweating agent 120 allowing a predetermined volume of perspiration to collect therein. That is, the sweating agent 120 may be designed to induce perspiration and pass the perspiration to the reservoir, but retain an amount of perspiration relatively less than contained in the reservoir. A hydrophilic layer may be interposed external to the sweating agent 120 to draw the perspiration into the reservoir. Additionally, a portion of the housing 110 (e.g., sides, the external face, etc.) may be perforated or formed from a mesh, allowing the perspiration to evaporate eventually.

In one exemplary embodiment, a detection arrangement, e.g., electrical connectors 125, engage and pass an electrical current through the sweat harvested by the device 100. As understood by those of skill in the art, flow of the electrical current between the electrical connectors 125 may depend on a concentration of salt(s) (e.g., sodium, potassium, etc.) in the sweat absorbed by the sweating agent 120. Thus, if the electrical connectors 125 engage the sweating agent 120 to pass the electrical current through the sweat absorbed therein, a resistance of the material forming the sweating agent 120 may have to be taken into account when determining the conductivity of the sweat.

In another exemplary embodiment, the detection arrangement may be a sensor or another measuring device may be utilized for determining an amount of one or more contents (e.g., sodium, chloride, potassium, electrolytes, alcohol, minerals, etc.) of the sweat.

The electrical connectors 125 may be powered by a power source, e.g., a battery 130, which may be removable or integrally formed in the housing 110. The battery 130 may be an alkaline battery or a rechargeable battery (e.g., Li ion cell). In the latter case, the battery 130 may be recharged via, for example, a physical coupling to an external power source, a solar cell on the device 100 or inductive charging.

In the exemplary embodiment, the battery 130 may also power a feedback arrangement 135. The feedback arrangement 135 may be disposed on an external face of the housing 110 such that it is visible to the user or an administrator. For example, a user using the device 100 for individual purposes may place the device 100 on the back of his hand or forearm. In, for example, a clinical setting, medical personnel may place the device 100 on any part of the body, which may not necessarily be easily visible by the user. For example, the device 100 may be applied to a user's underarm. As will be described further below, the feedback arrangement 135 may be any one or a combination of a light-emitting diode (LED), a liquid crystal display (LCD), an audible alarm, a mechanical alarm (e.g., vibration), wireless communication arrangement (e.g., transmitter, transceiver), etc.

In one exemplary embodiment, the feedback arrangement 135 may include the battery 130 and be removably coupleable to the housing 110 via, for example, a snap-fit, frictional fit, etc. For example, the feedback arrangement 135 may be a removable LCD disc which includes the battery 130 and an electrical contacts exposed on its internal face. In this embodiment, when the LCD disc is coupled to the housing 110, the electrical contacts engage the electrical contacts 125, allowing the device 100 to analyze the content(s) in the sweat. In a further exemplary embodiment, the feedback arrangement 135 may include the sensor, described above. In this embodiment, the housing 110 may be applied to the user's skin and the feedback arrangement 135 may be selectively coupled to the housing 110 to analyze the content(s) of the user's perspiration. Thus, if the adhesive layer 115 begins to fail, the feedback arrangement 135 may be removed while the housing 110 is replaced, and the feedback arrangement 135 can be coupled to a new housing.

The battery 130 may also provide power to a processing unit 140. The processing unit 140 may be embodied in hardware, software or any combination thereof and may include and/or have access to a memory or any data storage arrangement. In the exemplary embodiment, the processing unit 140 computes a value based on an amount of a preselected content(s) of the sweat in the sweat harvested by the device 100 and provides feedback via the feedback arrangement 135 regarding a concentration of the content(s). For example, in the exemplary embodiment, the processing unit 140 may determine the conductivity of the sweat and provide feedback indicative of the hydration level of the user. While the exemplary embodiments of the invention generally relate to producing feedback regarding the hydration level of the user, those of skill in the art will understand that the device 100 may generate feedback regarding other contents of sweat, e.g., electrolytes, minerals, alcohol, etc. The feedback in these other exemplary embodiments may indicate that the user should ingest (or be infused with) electrolyte-rich fluids, that the user is vitamin/mineral deficient, that the user is inebriated, etc. The feedback may also indicate that one or more fluids detected in the sweat were unknown or should not have contained therein.

In an exemplary use of the device 100 to determine a hydration level of the user, the device 100 may be applied to the user's skin as described above, e.g., via the adhesive layer 115, a wearable article, etc. The sweating agent 120 disposed on the housing 110 may induce the user's endocrine system to begin producing sweat, which is absorbed by the sweating agent 120. As the sweating agent 120 absorbs the sweat, a circuit is completed between the electrical connectors (e.g., the electrical current begins to pass between the electrical connectors 125) or the sensor analyzes the sweat. The processing unit 140 determines a conductivity of sweat by, for example, computing the electrical current passing between the electrical connectors 125 or receiving a data signal from the sensor indicative of the amount of salt in the sweat. The computed electric current may be compared to an index of electric current values which are associated with hydration levels. For example, the computed electric current value may be substantially equivalent to an entry in the index corresponding to an 85% hydration level, which can be indicated to the user via the feedback arrangement 135. As understood by those of skill in the art, the index includes a plurality of values which can be associated with, for example, varying levels of a predetermined content(s) of the sweat (e.g., sodium, potassium chloride, alcohol, minerals, etc. For example, when the device 100 is utilized to determined the hydration level of the user, ranges of the values in the index may indicate dehydration, optimal hydration and/or over-hydration.

In another exemplary embodiment, the computed electrical current may be compared to a threshold value which is an electric current value that represents an upper limit of salt concentration in the sweat for which the user may still be considered hydrated, e.g., will not experience symptoms associated with dehydration. When the computed electrical current is greater than the threshold value, the processing unit 140 may control the feedback arrangement 135 to provide feedback to the user. For example, when the feedback arrangement 135 is an LED, the processing unit 140 may activate the LED (e.g., illuminate, illuminate to a predetermined color or intensity, blink at a predetermined interval, etc.) to alert the user that he is or may begin experiencing the symptoms associated with dehydration if fluids are not ingested or introduced.

Those of skill in the art will understand that various types of feedback may be provided via the feedback arrangement 135. That is, the feedback arrangement 135 may be a two-state LED which, when activated (“on”), may indicate that the user is or may become dehydrated. In another exemplary embodiment, the feedback arrangement 135 may be an LCD which provides a set of feedback data including, for example, at least one of a current (updatable) hydration level of the user, the threshold value, a recommended fluid intake based on the hydration level, etc. As stated above, other exemplary embodiments of the feedback arrangement 135 may include an audible alarm or mechanical alarm which are activated by the processing unit 140 when the computed electrical current is greater than the threshold value.

The threshold value and/or the index may be computed by the processing unit 140 on a user-specific basis or may be pre-programmed. For example, the device 100 may include a communication device 150, e.g., a mini-USB port, a wireless transceiver, an RFID tag, etc. which allows the processing unit 140 to transmit the feedback to a remote device and/or receive data/instructions (e.g., an updated threshold value). The communication device 150 may be removably coupled to the device 100. The processing unit 140 may communicate user data with a remote computing device, or vice-versa, allowing either the processing unit 140 or the remote computing device to compute the threshold value and/or the index. The user data may include, for example, a height, weight, age, activity level, medical condition(s), etc. of the user.

If the threshold value or index is pre-programmed, it may be computed (e.g., from the user data) for a specific user and hard-coded on the processing unit 140 or may be computed for a preselected demographic bucket, e.g., males, 200-225 pounds, ages 25-30.

The index may be stored in the memory accessible by the processing unit 140. When the device 100 is utilized to determine a hydration level of the user, the index of values may be calibrated to include data corresponding to sodium concentration levels in the sweat. The sodium concentration levels may be associated with a hydration level. By comparing the value determined by the processing unit 140 from the user's sweat to the values in the index, the processing unit 140 may determine the sodium concentration level in the user's sweat, and thus, the user's hydration level, which may be output on the feedback arrangement 135. As understood by those of skill in the art, similar calibrations may be performed to determine an amount of alcohol, mineral(s), etc. in the sweat.

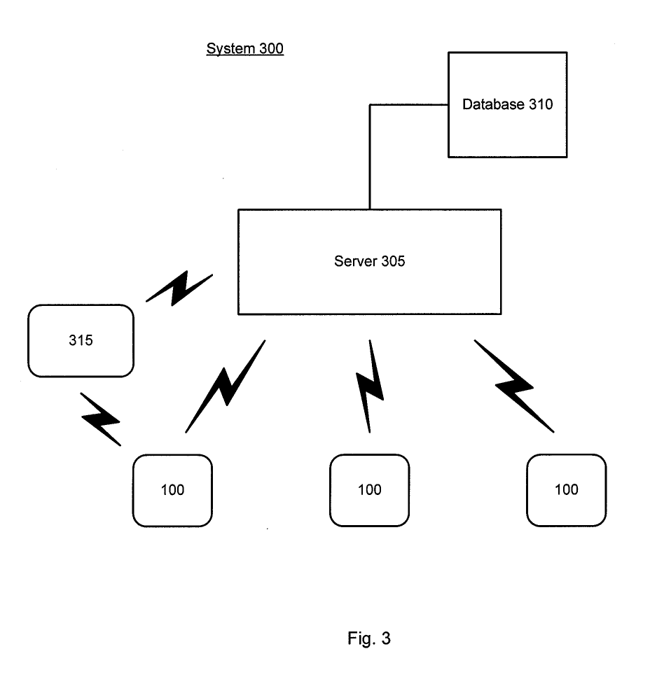

As shown in FIG. 3, an exemplary embodiment of a system 300 according to the present invention may include a remote computing device, e.g., a server 305, communicating with a plurality of the devices 100. This embodiment may be useful in, for example, a clinical setting in which the device 100 is attached to each of a plurality of users. Accordingly, each of the devices 100 may include a unique identifier which becomes associated with a patient to allow the server 305 to distinguish between signals received from the devices 100. The server 305 may allow medical personnel to receive and analyze the feedback from each of the devices 100 to monitor the hydration levels of the respective users. The server 305 may also be used to write data to the devices 100.

When communicating with the server 305, data packets transmitted by each of the devices 100 may include a unique identifier which allows the server 305 to segregate the data from a particular arrangement 100. For example, in the embodiment in which the communication devices 150 are wireless transceivers (e.g., 802.11x chipsets), data packets transmitted by each of the devices 100 may include a MAC address uniquely identifying each of the devices 100.

The server 305 may store and/or process the data received from the devices 100. For example, the server 305 may have access to a database 310 which includes data corresponding to each of the users. In the clinical setting, for example, the database 310 may include a given user's medical records, treatment protocols, medications and the data received from the device 100 worn by the user. Based on this data, the server 305 may update the user's medical records and/or treatment protocols, suggest modified dosages of the medications and/or fluids, etc. In another exemplary embodiment, the server 305 may transmit a signal to a remote device 315, activating/de-activating or adjusting operation thereof. For example, the remote device 315 may be a servo system, e.g., an infusion pump or intravenous fluid administration device, which infuses fluid into the user. If the server 305 receives the feedback indicating that a given user is dehydrated, the server 305 may send an instruction signal to the remote device 315 to increase the volume of fluid being supplied to the user.

In another exemplary embodiment, the remote device 315 may be an ignition device in a motor vehicle. For example, the device 100 may determine that the user is inebriated and the instruction signal may disable the ignition device until a predetermined procedure has taken place (e.g., a pass key is entered) or for a predetermined amount of time.

In another exemplary embodiment, the communication device 150 on the device 100 may communicate directly with the remote device 315. For example, the communication device 150 may transmit a wireless signal (e.g., 802.11x, Bluetooth, etc.) periodically (or asynchronously) to the remote device 315. The remote device 315 may include a processor which interprets the feedback and adjusts its operation as a function thereof. In the above example, the communication device 150 may send a signal to the remote device 315 indicating that the user is dehydrated. The remote device 315 may adjust its operation and/or send a signal to the server 305 relaying the feedback received from the device 100. This exemplary embodiment may provide a notification mechanism whereby medical personnel can monitor operation of the remote device 315 and the medical condition of the users.

In another exemplary embodiment, the communication device 150 may allow the device 100 to be established as part of a personal area network (PAN). In this embodiment, the device 100 may communicate data with a wireless headset or handheld computing device (e.g., cell phone, PDA, etc.) to provide the feedback.

Notably, the figures and examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention. In the present specification, an embodiment showing a singular component should not necessarily be limited to other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the known components referred to herein by way of illustration.

The foregoing description of the specific embodiments will so fully reveal the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present invention have been described above, it should be understood that they have been presented by way of example, and not limitation. It would be apparent to one skilled in the relevant art(s) that various changes in form and detail could be made therein without departing from the spirit and scope of the invention. Thus, the present invention should not be limited by any of the above-described exemplary embodiments, but should be defined only in accordance with the following claims and their equivalents.

What is claimed is:

1. A device, comprising:
a memory storing an index of values corresponding to amounts of a content in perspiration;
a detection arrangement detecting the content in the perspiration;
a processing unit determining an amount of the content in the perspiration; and
a feedback arrangement providing feedback data indicative of a value corresponding to the determined amount.

2. The device according to claim 1, further comprising:
a sweating agent inducing the perspiration when applied to skin.

3. The device according to claim 2, wherein the sweating agent is pilocarpine.

4. The device according to claim 2, further comprising:
a housing having first and second faces, the sweating agent being disposed on the first face.

5. The device according to claim 4, wherein the second face includes an engagement arrangement removably engaging at least one of the memory, the processing unit and the feedback arrangement.

6. The device according to claim 1, further comprising:
an adhesive coupling the device to skin.

7. The device according to claim 1, wherein the detection arrangement includes electrical contacts delivering a current to the perspiration, the processing unit computing a conductivity of the perspiration as a function of the current.

8. The device according to claim 7, wherein the values correspond to conductivities and hydration levels.

9. The device according to claim 8, wherein the feedback data indicates the hydration level.

10. The device according to claim 1, wherein the detection arrangement includes a sensor.

11. The device according to claim 10, wherein the sensor transmits a signal to the processing unit indicative of the amount of the content.

12. The device according to claim 1, further comprising:
a power source.

13. The device according to claim 12, wherein the power source is one of an alkaline battery, a rechargeable battery and a solar cell.

14. The device according to claim 1, wherein the feedback arrangement includes at least one of a liquid crystal display, a light-emitting diode, an audible alarm, a mechanical alarm and a wireless communication arrangement.

15. The device according to claim 14, wherein the feedback data of the wireless communication arrangement is included in a wireless signal transmitted thereby.

16. The device according to claim 1, wherein a predetermined value from the index of values is a threshold value.

17. The device according to claim 16, wherein the feedback arrangement provides further feedback data indicating that the value is one of greater and less than the threshold value.

18. The device according to claim 1, wherein the content is one of a salt, an alcohol and a mineral.

19. A system, comprising:
a computing device; and
a wearable measuring device measuring an amount of a content of perspiration and transmitting feedback data indicative of the amount to the computing device.

20. The system according to claim 19, wherein the wearable measuring device includes a memory storing an index of values corresponding to amounts of a content in perspiration, a detection arrangement detecting the content in the perspiration and a processing unit determining an amount of the content in the perspiration and generating the feedback data as a function of the amount.

21. The system according to claim 19, wherein the computing device is one of a server, a mobile phone, a PDA, a portable media player, a portable gaming device and a handheld computer.

22. The system according to claim 19, further comprising:
a database storing the feedback data.

23. The system according to claim 19, further comprising:
a servo system, wherein one of the computing device and the wearable measuring device transmit the feedback data to the servo system and the servo system executes a predetermined operation upon receipt of the feedback data.

24. The system according to claim 23, wherein the servo system is one of an intravenous fluid delivery system and a vehicle ignition system.

25. The system according to claim 19, wherein the content is one of a salt, an alcohol and a mineral.

26. The system according to claim 19, wherein the wearable measuring device includes a unique identifier and transmits the unique identifier to the computing device.

27. The system according to claim 26, wherein the computing device associates the feedback data with the unique identifier.

28. A device, comprising:
a memory storing a first value corresponding to a conductivity of perspiration;
a power source supplying a current to the perspiration;
a processing unit generating a second value as a function of the current, the processing unit comparing the first and second values to determine a hydration level; and
a feedback arrangement providing feedback indicative of the hydration level.

29. The device according to claim 28, wherein the feedback is one of a visual indicator and an audible indicator.

30. The device according to claim 28, further comprising:
a housing encasing the memory, the power source, the processing unit and the feedback arrangement.

31. The device according to claim 30, further comprising:
a sweating agent disposed on the housing to induce the perspiration.

32. A device, comprising:
a memory storing a first value corresponding to a predetermined amount of a content of perspiration;
a sensor measuring an amount of the content of the perspiration;
a processing unit generating a second value as a function of the measured amount, the processing unit comparing the first and second values to determine a hydration level; and
a feedback arrangement providing feedback indicative of the hydration level.

33. The device according to claim 32, wherein the feedback is one of a visual indicator and an audible indicator.

34. The device according to claim 32, further comprising:
a housing encasing the memory, the sensor, the processing unit and the feedback arrangement.

35. The device according to claim 34, further comprising:
a sweating agent disposed on the housing to induce the perspiration.
